(19) United States
(12) Patent Application Publication
(10) Pub. No.: US 2023/0278976 A1
(43) Pub. Date: Sep. 7, 2023

(54) Synthesis Of Beta-Glycolipid Compounds

(71) Applicant: Glycosurf, Inc., Salt Lake City, UT (US)

(72) Inventors: Chett Boxley, Park City, UT (US); Ryan Stolley, Salt Lake City, UT (US); Robert Bruggeman, Salt Lake City, UT (US)

(21) Appl. No.: 17/858,059

(22) Filed: Jul. 5, 2022

Related U.S. Application Data
(60) Provisional application No. 63/218,424, filed on Jul. 5, 2021.

(51) Int. Cl. C07D 309/10

(52) U.S. Cl. CPC C07D 309/10

(57) ABSTRACT

Methods of forming a glycolipid are disclosed. A method may include glycosylating a sugar with a beta-hydroxyester using BF3-L as a glycosylation promoter. L may include one or more of Diethylether, Tetrahydrofurn, Dimethyle Sulfide, or combinations thereof. The beta-hydroxyester may include a carbon length ranging from C2 to C24. The sugar may be a protected sugar that includes peracetylated-rhamnose, peracetylated-xylose, peracetylated-glucose, peracetylated-galactose or combinations thereof. The beta-hydroxyester and sugar may be combined in a reaction vessel to form a mixture. A solvent may be added to the mixture to substantially dissolve the mixture. The glycosylation promoter may be added to the solution such that the solution is maintained withing a desired temperature range.

### CROSS-REFERENCE TO RELATED APPLICATIONS

This application claims the priority benefit of U.S. Provisional Application No. 63/218,424, filed Jul. 5, 2021, which is incorporated herein by reference in its entirety.

### STATEMENT REGARDING FEDERALLY FUNDED RESEARCH

This invention was made with government support under Grant No. 2R44ES031897-02 awarded by the National Institute of Environmental Health Science. The government has certain rights in the invention.

### TECHNICAL FIELD

The application relates generally to the synthesis of beta-glycolipid products, and more specifically, to methods of synthesizing beta-glycolipid products from either natural or synthetic beta-hydroxy esters or beta-hydroxy carboxylic acids using boron trifluoride complexes.

### BACKGROUND

Beta-glycolipid are used as an active ingredient in many commercial products. The problem with current methods of synthesizing Beta-glycolipids are that the agent used to promote the combination or reaction of a carbohydrate with a lipid is very expensive even though it is used in sub-stoichiometric quantities. Additionally, present methods of synthesizing glycolipids do not lend themselves to scaling production beyond a few grams to tens of grams. The current methods have too many variables and too many process conditions that are hard to control when trying to mass produce glycolipids. Additionally, current processes don't create a very high yield of glycolipids. Thus, is takes more reactants and more costly and challenging processing conditions in order to achieve a small amount of material, which significantly increases production costs.

What is needed are more cost-effective methods of synthesizing glycolipids, where a cheaper glycosylation promoter may be used with lower amounts of reactants to create a higher yield of glycolipid products. Additionally, it would be an approvement over the prior art to have a process with process conditions that are easier to control so that the process may be scaled for mass production. Such a process is described and claimed herein.

### SUMMARY

Embodiments are disclosed herein regarding the synthesis of beta-glycolipid products. The method described allows for the synthesis of a broad array of beta-glycolipid products using one of many different carbohydrate structures possible. Also described is the synthesis using either natural or synthetic beta-hydroxy esters or beta-hydroxy carboxylic acids having various chain lengths and varying degrees of saturation. The synthesis may utilize boron trifluoride complexes as the inexpensive glycosylation promoter. In one embodiment, the boron trifluoride complex is BF3-L where L includes one or more of diethyl ether (Et2O), tetrahydrofuran (THF), dimethyl sulfide (Me2S), methanol (CH3OH), acetonitrile (CH3CN), acetic acid (CH3CO3H), water (H2O), a variety of amines or combinations thereof. In one embodiment, the beta-hydroxy esters or acids may be derived from metal enolates as lipid congeners. In one embodiment, the metal enolates may include lithium or zinc. In one embodiment the beta-hydroxy acids or esters are natural products.

### BRIEF DESCRIPTION OF THE SEVERAL VIEWS OF THE DRAWINGS

FIG. 1 is a flowchart showing Rhamnose acetylation;

FIG. 2 is a flowchart showing Tail Group Synthesis;

FIG. 3 is a flowchart showing Rhamnose peracetate glycosylation to make a single chain product;

FIG. 4 is a flowchart showing glycosylation to make a double chain product;

FIG. 5 is a flowchart showing glycolipid global deprotection; and

FIG. 6 is a table showing process configuration options utilizing FIGS. 1-5.

### DETAILED DESCRIPTION

In the following detailed description, reference is made to the accompanying drawings which form a part hereof, and in which is illustrated specific embodiments in which the disclosure may be practiced. These embodiments are described in sufficient detail to enable those of ordinary skill in the art to practice the disclosure. It should be understood, however, that the detailed description and the specific examples, while indicating examples of embodiments of the disclosure, are given by way of illustration only and not by way of limitation. For example, The detailed description includes various embodiments of the compositions and methods of the present invention. These embodiments are described in sufficient detail to enable those of ordinary skill in the art to practice the disclosure. However, before the present materials and methods are described, it is to be understood that this invention is not limited to the particular molecules, compositions, active ingredients, methodologies, or protocols herein described, as these may vary in accordance with routine experimentation and optimization. It is also to be understood that the terminology used in the description is for the purpose of describing the particular versions or embodiments only and is not intended to limit the scope of the embodiments described herein. Accordingly, various substitutions, modifications, additions rearrangements, or combinations thereof are within the scope of this disclosure. Furthermore, all or a portion of any embodiment disclosed herein may be utilized with all or a portion of any other embodiment, unless stated otherwise.

In accordance with common practice, the various features illustrated in the drawings may not be drawn to scale. The illustrations presented herein are not meant to be actual views of any particular composition, molecule, ingredient, or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given embodiment or all operations of a particular method. In addition, like reference numerals may be used to denote like features throughout the specification and figures. Furthermore, all or a portion of any embodiment disclosed herein may be utilized with all or a portion of any other embodiment, unless stated otherwise.

In addition, it is noted that the embodiments may be described in terms of a process that is depicted as method steps, a flowchart, a flow diagram, a schematic diagram, or a block diagram. Although a flowchart, process, or method may describe operational acts as a sequential process, many of these acts can be performed in another sequence, in parallel, or substantially concurrently. In addition, the order of the acts may be re-arranged. A process may correspond to a method, a function, a procedure, a subroutine, a subprogram, etc.

The terms used in describing the various embodiments of the disclosure are for the purpose of describing particular embodiments and are not intended to limit the disclosure. As used herein, the singular forms are intended to include the plural forms as well, unless the context clearly indicates otherwise. All of the terms used herein including technical or scientific terms have the same meanings as those generally understood by an ordinary skilled person in the related art unless they are defined otherwise. Terms defined in this disclosure should not be interpreted as excluding the embodiments of the disclosure. Additional term usage is described below to assist the reader in understanding the disclosure.

The terms “have,” “may have,” “include,” and “may include” as used herein indicate the presence of corresponding features (for example, elements such as numerical values, functions, operations, or ingredient), and do not preclude the presence of additional features.

The word “exemplary” is used herein to mean “serving as an example or illustration.” Any aspect or design described herein as “exemplary” is not necessarily to be construed as preferred or advantageous over other aspects or designs.

The terms “A or B,” “at least one of A and B,” “one or more of A and B”, or “A and/or B” as used herein include all possible combinations of items enumerated with them. For example, use of these terms, with A and B representing different items, means: (1) including at least one A; (2) including at least one B; or (3) including both at least one A and at least one B. In addition, the articles “a” and “an” as used herein should generally be construed to mean “one or more” unless specified otherwise or clear from the context to be directed to a singular form.

Terms such as “first,” “second,” and so forth are used herein to distinguish one component from another without limiting the components and do not necessarily reflect importance, quantity, or an order of use. For example, a first washing and a second washing may indicate different washings regardless of the order or importance. Furthermore, a reference to first and second elements does not mean that only two elements may be employed there or that the first element must precede the second element in some manner. Also, unless stated otherwise a set of elements may comprise one or more elements.

The section headings provided herein are for convenience only do not interpret the scope or meaning of the claimed options. Furthermore, unless otherwise defined, all technical and scientific terms used herein have the samemeaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

The expression “configured to” as used herein may be used interchangeably with “suitable for,” “having the capacity to,” “designed to,” “adapted to,” “made to,” or “capable of” according to a context. The term “configured” does not necessarily mean “specifically designed to,” and the expression compound or composition “configured to . .” may mean that the compound or composition is “capable of . . . ” along with other compounds or compositions in a certain context.

Unless the context otherwise requires, in the description text and in the claims that follow, the term “contain” and its derivatives, such as “contains” and “containing,” should be considered open, non-restrictive forms, that is, as “including but not limiting.” In addition, the terms “having,” or “including” should be understood as “including but not limited to the specific member or members listed.

The term “about,” as used herein, includes any value that is within 10% of the described value.

The term “between,” as used herein, is inclusive of the lower and upper number of the range.

Reference herein to any numerical range (for example, a dosage range) expressly includes each numerical value (including fractional numbers and whole numbers) encompassed by that range. For example, but without limitation, reference herein to a range of 13° C. to 33° C. explicitly includes all whole numbers and fractional numbers between the two.

The term “beta” or “beta-,” as used as a prefix with words such as glycolipid or hydroxy esters, and the like can be left off without changing the meaning of the combined term. In other words, the term “beta-glycolipid” and “beta glycolipid” can be used interchangeably with the term “glycolipid” and words or terms modified with the word “beta-” or “beta” can be used interchangeably with the word or term without “beta” or “beta-.”

The term “glycolipid” may be used synonymously with “glycol-lipid” and should not be interpreted to be limiting in any way to any particular type. The term “glycolipid” is meant to include its subgroups and groups in which it may be found including, without limitation, Glycoglycerolipids, Galactolipids, Sulfolipids, Sulfoquinovosyl Diacylglycerols, Glycosphingolipids, Cerebrosides, Galactocerebrosides, Glucocerebrosides, Sulfatides, Gangliosides, Globosides, Glycophosphosphingolipids, Phytoglycolipids, Glycophosphatidylinositols, and Saccharolipids.

The terms “formula” and “molecule” may be used interchangeably in certain contexts.

### EMBODIMENTS

Embodiments of this disclosure describe the synthesis of beta-glycolipid products using lipids that may be either natural or synthetic beta-hydroxy esters or synthetic beta-hydroxy carboxylic acids. In one embodiment, beta-hydroxy esters or acids synthesized using metal enolates may be utilized as lipid congeners for the synthesis of beta-glycolipds. The metal enolates in certain embodiments may be enolates of one or more of lithium, boron, zinc, sodium, potassium, nickel, or combinations of these. Accordingly, the beta-hydroxy esters or beta-hydroxy carboxylic acids used in this process may be synthesized. In other embodiments, the beta-hydroxy esters or beta-hydroxy carboxylic acids may be obtained from a natural source. In certain embodiments, one of the natural beta-hydroxy esters or natural beta-hydroxy carboxylic acids can be used to synthesize the other. The natural or synthesized beta-hydroxy esters or beta-hydroxy carboxylic acids may have a carbon chain length of between C6 and C24 inclusive. The natural sources of beta-hydroxy acids or esters may be obtained as a mixture of several chain length beta-hydroxy acids or esters, or they may be highly purified versions of a single chain length. Also, the natural sources of beta-hydroxy acids or esters may contain molecules of the same chain length, but with single, double, or poly unsaturation's in the chain length. The positions within the chain where the unsaturation (double bonds) occurs may vary depending on the natural source of the obtained material.

One or more of the lipids described above may be attached to a carbohydrate. The carbohydrate may be an acel-protected carbohydrate. The carbohydrate may be a sugar. In one embodiment, the sugar is a protected sugar. The lipid may be combined with the sugar using a glycosylation promotor. The glycosylation promoter may be a boron trifluoride complex, including without limitation, BF₃₋ L (where L=Et2O, THF, and/or Me2S). The general process of attaching or combining a lipid with a carbohydrate (including a sugar) using a glycosylation promotor may be referenced herein throughout as “glycosylation.”

In one embodiment, a method of synthesizing glycolipids includes the glycosylation of beta hydroxy esters using BF₃₋ L (where L=Et2O, THF, Me2S) as the glycosylation promoter. In one embodiment, the b-hydroxy esters have a carbon chain length of between about C2 and about C24. In other embodiments, the length is between about C8 and about C18. In yet another embodiment, the length is between about C10 and about C16. The beta hydroxy ester in one embodiment is derived from natural source. In another embodiment, the beta hydroxy ester is synthesized as described hereafter. The beta hydroxyester may be combined with, or glycosylated, using a sugar. The sugar may be a mono-saccharide, di-saccharide, or tri-saccharide. The sugar may be one or more peracetylated-rhamnose, peracetylated-xylose, peracetylated-glucose, peracetylated-galactose, peracetylated melibiose, peracetylated cellobiose, peracetylated fucose, or combinations thereof. This embodiment may be illustrated by the following reaction:

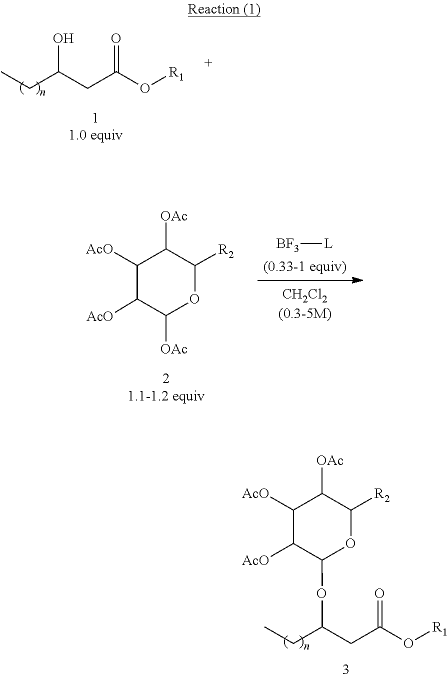

where R₁=Me, Et, Bn, MOM, and/or MEM, R₂=2H, Me, and/or CH₂OAc, L=Et₂O, THF, and Me₂S, and n is between 2 and 24 inclusive.

In Reaction 1 above, it will be appreciated that the equivalents listed for R₁, R₂, and L are molar equivalents. Thus, the embodiments of the present invention can be said to describe stoichiometric reactions. This differs from prior art reactions that are more catalytic. In one embodiment, the lipid molecule identified as “1” in the reaction above may have an equivalent of about 1.0. This molecule and its equivalents may be referred to as “molecule 1”. The sugar molecule identified as “2” may have an equivalent of between about 1.1 and about 1.2. This molecule and its equivalents may be referred to as “molecule 2”. The glycosylation promotor on the reaction arrow may have an equivalent of between about 0.33 and about 1.0. In other embodiments, the glycosylation promotor on the reaction arrow may have an equivalent of between about 0.33 and about 1.3. The solvent in this embodiment may be CH₂Cl₂ in an amount of between about 0.3 and about 5M. The reaction creates a glycolipid identified as “3” in the reaction, which may be referred to as “formula 3”.

In one embodiment, molecule 1 may be any lipid having the formula:

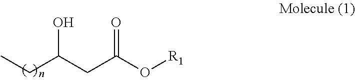

In one embodiment, R₁ may be a functional group including one or more of a methyl functional group (Me), an ethyl functional group (Et), a benzyl functional group (Bn), a methoxymethyl functional group (MOM), a methoxyethoxymethyl functional group (MEM), or combinations thereof. The lipid molecule may have an equivalent of about 1.0.

Molecule 2 may be any sugar having the formula:

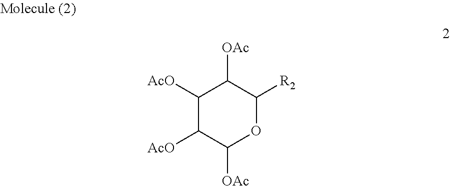

In one embodiment, R₂ may be a pyranosyl methylene group (2H), a methyl functional group (Me), a CH₂OAc group, or combinations thereof. In another embodiment, R₂ may be any dioxy glycol puano glycan. In one embodiment, when rhamsnose or xylose is used as the sugar, the equivalent of the glycosylation promoter is about 0.35. In other embodiments, when the sugar glucose or galactose, the glycosylation promoter equivalent is closer to about 1.3. The sugar molecule itself may have an equivalent of between about 1.1 and about 1.2.

The glycosylation promotor may a boron trifluoride complex BF₃₋ L. In one embodiment, L represents one or more of Diethyl ether (Et₂O), Tetrahydrofuran (THF), Dimethyle Sulfide (Me₂S), or combinations thereof. In other embodiments, L may be any Lewis based adduct to boron trifluoride.

In one embodiment, the end product (3) may be any protected glycolipid having the formula:

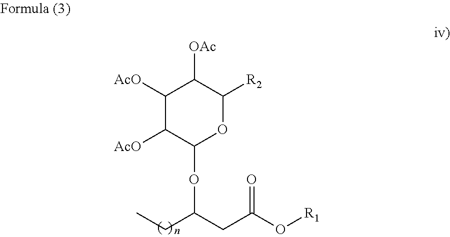

In one embodiment of the process of synthesizing a glycolipid, the sugar is a pyranoside. In other embodiments, the sugar may be a furanose. In other embodiments, the sugar may be a deoxysugar. In yet another embodiment, the sugar may be one or more of the protected forms of the sugars listed in Appendix A. The sugar may have one or more of the structures listed in Appendix B. In certain embodiments, the lipid may have one or more of the structures listed in Appendix C. Appendices A, B, and C are incorporated herein in their entirety by this reference.

One embodiment of synthesizing a glycolipid includes mixing a beta hydroxyester and a peracetylated sugar in an adequately-sized reaction vessel. In one embodiment, the mixing is accomplished using a stirring device. By way of non-limiting example, the stirring device may be a magnetic stirbar, mechanical stirrer, a paddle mixer, and the like. In one embodiment, the mixing may be accomplished by a stirring or mixing device that is part of the reaction vessel. In other embodiments, the mixing may be done with devices that are separate from the reaction vessel. The mixing may be accomplished automatically as part of an automated system when certain threshold measurements are achieved. These measurements may include a weight, a saturation level, an amount of one or more added materials, a time, a temperature, a pH level, a pressure, and the like. Sensors, known in the art to detect and/or measure such measurements may be used to determine a threshold. A computer processor may control the mixing process and communicate with one or more sensors to determine when to start and/or stop the mixing step.

The mixing step may continue until the b-hydroxyester and the peracetylated sugar are substantially mixed. In one embodiment, the b-hydroxyester and/or the peracetylated sugar is uniformly distributed throughout at least about 70% of the total mixture. In another embodiment, the b-hydroxyester and/or the peracetylated sugar is uniformly distributed throughout at least about 80% of the total mixture. In yet another embodiment, the b-hydroxyester and/or the peracetylated sugar is uniformly distributed throughout at least about 90% of the total mixture.

In one embodiment of the method, the atmosphere within the reaction vessel containing the reactants may be purged to create an inert atmosphere. This may be accomplished using N₂, Ar, or other materials known in the art to achieve an inert atmosphere.

The method may include adding a solvent to the mixture to create a solution. More than about 1 mL/g of solvent may be mixed with the mixture. In another embodiment, less than about 10 mL/g may be mixed into the mixture. In yet another embodiment, between about 3 mL/g and 7 mL/g may be mixed into the mixture. In yet another embodiment, about 5 mL/g of solvent may be mixed into the mixture. The method may include adding the solvent to the mixture to form a solution wherein the mixture is more than 70% dissolved in the solvent. In one embodiment, the mixture is more than 80% dissolved in the solvent. In one embodiment, the mixture is more than 90% dissolved in the solvent. In one embodiment, the mixture is completely dissolved in the solvent. Dissolution of the mixture in the solvent may be measured or determined manually or automatically using ways known in the art, include without limitation, thin layer chromatography, gas chromatography, high-performance liquid chromatography, and the like. In one embodiment, the solvent is dry dichloromethane.

The method may include adding a glycosylation promoter to the solution. The glycosylation promoter may be the BF₃₋ L described above. The glycosylation promoter may be added to the mixture such that the resulting reaction product and/or the solution temperature is within a predetermined range. In one embodiment, the predetermined range is between about 13° C. and about 33° C. inclusive. In yet another embodiment, the predetermined range is between about 16° C. and about 30° C. inclusive. In yet another embodiment, the predetermined range is between about 19° C. and about 27° C. inclusive. In yet another embodiment, the predetermined range is between about 22° C. and about 24° C. inclusive. In yet another embodiment, the predetermined range is about room temperature.

Adding the glycosylation promoter to the solution may include slowly adding the solvent. In other embodiments, the solvent may be added all at once. In one embodiment, adding a solvent to the mixture may include stirring the reaction product in an inert atmosphere until the b-hydroxyester is at least 70% consumed. In another embodiment, the method may include stirring the reaction product in an inert atmosphere until the b-hydroxyester is at least 80% consumed. In yet another embodiment, the method may include stirring the reaction product in an inert atmosphere until the b-hydroxyester is at least 90% consumed. In one embodiment, the method may include stirring the reaction product in an inert atmosphere until the b-hydroxyester is completely consumed. Consumption of the b-hydroxyester may be measured or determined manually or automatically using ways known in the art, include without limitation, thin layer chromatography, gas chromatography, high-performance liquid chromatography, and the like. In one embodiment, the reaction product is stirred for at least 4 hours. In another embodiment, the reaction product is stirred at least 5 hours. In another embodiment, the reaction product is stirred less than about 20 hours. In another embodiment, the reaction product may be stirred between about 6 and about 12 hours. The stirring may be done in an inert atmosphere.

The method may include the step of diluting the glycosylation reaction described herein by doubling the volume of solvent, which may be dichloromethane. The method may include the step of adding saturated sodium bicarbonate or another quenching compound to the diluted mixture of BF₃₋ L and solution to quench one or more of the glycosylation reaction, the reaction within the reaction vessel, and any reaction occurring as the result of any of the previous steps. Other materials may be used to quench reactions, including without limitation, other mildly basic aqueous bases including alkali metal carbonates, dilute alkali metal hydroxides, aline earth carbonates or oxides, or other moderately basic related aqueous inorganic bases. In one embodiment, the saturated sodium bicarbonate is added slowly. The sodium bicarbonate may be added such that uncontrolled bubbling or frothing is prevented. In one embodiment saturated sodium bicarbonate or another quenching compound is added until the release of CO₂ ceases. In one embodiment, the quenching compound until the pH of the solution is between about 7.5 and about 9. The quenching compound may be added in several steps over a period of time. It will be appreciated by those of skill in the art that other quenching methods may be used.

The method may include the step of removing an aqueous layer from the resulting material after the reaction has been quenched. In one embodiment, the remaining material is an organic solution. The method may include washing the remaining material with deionized water or liquid that is substantially ion free to maintain product purity. In one embodiment, the remaining material is washed at least one time. In another embodiment, the remaining material is washed less than four times.

In one embodiment, a drying agent may be added to the washed remaining material. The drying agent may be an agent that does not react with the remaining material. In one embodiment, the drying agent may be a brine solution. The combination of brine solution and remaining material may be dried over anhydrous Na2SO4 or MgSO4. The resulting dried material may be filtered and/or concentrated. Filtering, concentrating, and/or drying may be done under vacuum. The resulting product may be purified by flash chromatography or other purification methods know in the art. In one embodiment, purification may be accomplished by flash chromatography using 25% EtOAc/Hexanes. In other embodiments, the purification step may be omitted. In one embodiment, the glycosylation of b-hydroxy esters using BF₃₋ L as the glycosylation promoter produces a glycolipid yield of between about 65% and about 95%.

In one embodiment, a process may use a synthesized lipid where beta hydroxy esters and/or acids are synthesized from metal enolates (M=Li and/or Zn) as lipid congeners as shown below:

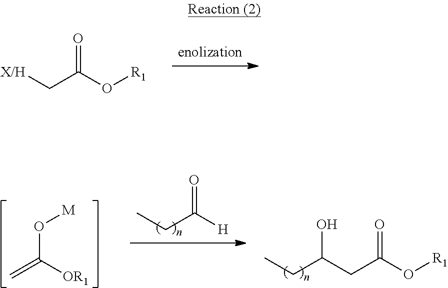

where R₁=Me, Et, Bn, MOM, and/or MEM, and M=Li, and/or Zn. The procedure of this embodiment may include step wise preparation of an ester enolate, via deprotonation, followed by addition of a long-chain aldehyde. Temperature and atmosphere maintenance are part of the process or method.

In one embodiment, the method of synthesizing hydroxy esters and/or acids may be part of the method of synthesizing glycolipids. This step may be a pre-step before the glycosylation process described above. This method or method pre-step may include purging a reaction vessel with N₂, Ar, or other purging material know in the art to achieve an inert atmosphere. As discussed above, the reaction vessel may include various types of stirring or mixing mechanisms.

A dry ethereal solvent may be added to the reaction vessel. The solvent may include, by way of non-limiting example, one or more of Et₂O, THF, 2-Me-THF, or combinations thereof. The amount of solvent added may be between about 0.3 and about 0.5M. The amount of solvent may control the reaction concentration. In one embodiment, either prepared Lithium diisopropylamine (LDA) or a combination of diisopropylamine and n-butyllithium may be added to the solvent. In one embodiment that amount of such material added is 1.1 molar equivalents to the ester progenitor. If n-butyl lithium is added it may have an equivalent of about 1.1. The n-butyl lithium may be added to the reaction vessel in one portion via cannula. This may be followed by addition of disopropylamine. Alternative materials may be added to the solvent, in about a 1.1 molar equivalents to the limiting reagent that is being substituted. The active base in reactions such as Reaction (2) in certain embodiments is lithium disopropylamide (LDA). This may be made by deprotonating diisopropylamine with butyl lithium. This can be done by a supplier to provide preformed LDA or made in the method described above.

In certain embodiments, the O in Reactions (1) and/or (2) and in formulas and/or molecules (1)-(3) may be replaced with S and R₁ and/or R₂ in these Reactions, formulas and/or molecules may comprise an ether and/or a metal.

The reaction vessel and/or its contents may be cooled to a predetermined temperature or within a predetermined temperature range. In one embodiment, reaction vessel and/or its contents is cooled to at least −50° C. In other embodiment, the predetermined reaction vessel and/or its contents may be cooled to no more than −78° C. In one embodiment, the reaction vessel and/or its contents may be cooled down to at most a temperature just warmer than the temperature where the reaction vessel and/or its contents would precipitate due to being too cold. In another embodiment, the reaction vessel and/or its contents may be cooled to at least a temperature just colder the temperature where the reaction vessel and/or its contents would degrade due to not being cold enough.

Once cooled, Di isopropyl amine maybe added slowly to the reaction vessel and/or its contents to maintain the temperature in a predetermined range. Once the Di isopropyl amine is complete added, the contents of the reaction vessel may then be stirred for up to about 60 minutes. In another embodiment, after the addition of Di isopropyl amine is completed, the contents may be stirred at least about 10 minutes. In yet another embodiment, the contents are stirred for an additional 20 minutes. In one embodiment, the contents are stirred enough to afford LDA or another base used to form enolate anions. It will be appreciated that strong organic bases formed by processes of the present invention, such as LDA: (Lithium Di isopropyl Amide), may be used to drive the ketone-enolate equilibrium completely to the enolate side.

While maintaining the contents of the reaction vessel at the predetermined temperature or temperature range, the appropriate ester is added dropwise. Once the appropriate ester has been completely added, the contents of the reaction vessel may be stirred for up to an additional 60 minutes. In another embodiment, the contents of the reaction vessel may be stirred at least an additional 10 minutes after the addition of the appropriate ester is complete. In yet another embodiment, the contents of the reaction vessel may be stirred about 20 additional minutes after the addition of the appropriate ester is complete.

At this time, the appropriate aldehyde may then be added dropwise while maintaining the predetermined temperature or temperature range of the reaction vessel and/or its contents. Once the appropriate aldehyde has been completely added to the reaction vessel, the contents of the reaction vessel may be stirred for up to an additional 6 hours. In another embodiment, the contents of the reaction vessel may be stirred at least an additional 60 minutes after the addition of the appropriate ester is complete. In yet another embodiment, the contents of the reaction vessel may be stirred about 3 hours after the addition of the appropriate aldehyde is complete. The reaction may then be allowed to warm to about 0° C. This may happen over a period of up to 3 hours. The contents may then be stirred at this temperature for up to an additional 2 hours.

The reaction progress may be tracked for the consumption of aldehyde by appropriate chemical test (e.g., TLC, GC, HPLC). Upon completion, the reaction is quenched by the slow addition of saturated ammonium chloride solution and allowed to stir for at least 5 minutes and up to 60 minutes. In one embodiment, the resulting material is stirred until the reaction is quenched. The reaction may then be diluted with ethyl acetate and/or deionized water. Once the contents are thoroughly mixed and an aqueous layer separates. The aqueous layer is removed and the remaining organic fraction may then be washed with 1M HCl. It may then be washed up to three times with deionized water. A drying agent may then be added to the remaining material. In one embodiment, the drying agent is brine. The contents may then be dried over anhydrous Na2SO4 or MgSO4 and condensed under vacuum. The contents may be dried in other non-reactive ways know in the art. If the product is solid or semisolid, the remaining product may be dissolved in an appropriate amount of hot methanol or other suitable alcohol and precipitated at —up to 30° C. The methanol may then be condensed and the dissolving steps repeated until precipitation stops. The remaining product may be purified by flash chromatography. In one embodiment, the process produces a beta hydroxy ester yield of between about 75% and about 98% yield.

In another embodiment, lipids for use in synthesizing glycolipids according to embodiments of the present invention are made using a homogeneous Reformatsky reaction. A homogeneous protocol may be used to avoid scaling limitations caused by heterogeneous protocols. In one embodiment, a process of synthesizing lipids for use in making glycolipids uses a soluble zinc progenitor, Et₂Zn or Me₂Zn, with the appropriate halo acetate ester either alone or in combination with a nickel-catalyst. This nickel catalyst can be of the form (PR₃)NiX₂, (PR₃)4Ni, Ni(AcAc)2, Ni(OAc)2, or the formation of such catalysts in situ from the appropriate nickel and ligand source. In one embodiment, lipids are synthesized using the following reaction:

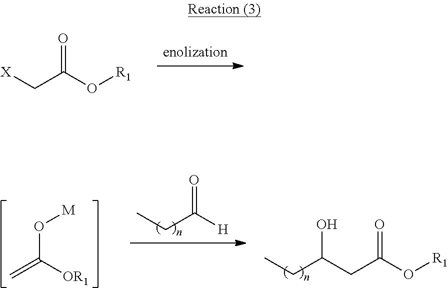

where R1=Me, Et, Bn, MOM, and/or MEM, M=Zn, and/or Ni, and X═Cl, Br, and/or I.

To a reaction vessel of adequate size is equipped with a stirring device (e.g., magnetic stirbar or mechanical stirrer). The reaction vessel is then purged with N₂ or Ar to achieve an inert atmosphere, followed by addition of dry and degassed CH2Cl2 (01 M total volume). To this reaction vessel is then added the appropriate haloactetate ester (1.1 equiv), the appropriate aldehyde (1.0 equiv) followed by addition of R2Zn (1.5 equiv) either neat or as a hydrocarbon solution. Separately, the appropriate catalyst is dissolved in dry and degassed CH2Cl2, and the resulting solution is added to the main reaction solution. Reaction (3) is allowed to proceed until complete consumption of the aldehyde is determined by appropriate chemical test (e.g., TLC, GC, HPLC).

Upon completion, this reaction is quenched by the slow addition of saturated ammonium chloride solution and allowed to stir for 15-30 min to ensure quenching. The reaction is then diluted with ethyl acetate and deionized water, thoroughly mixed and the aqueous layer separated. Due to the formation of insoluble zinc salts, settling or filtering at this time may be needed. The organic fraction is then successively washed with 3×deionized water and brine then dried over anhydrous Na2SO4 or MgSO4 and condensed under vacuum. If the product is solid or semisolid, the crude product is dissolved in a minimal amount of hot methanol and precipitated at −30° C. Nickel impurities can be removed by dissolving the product in ethyl acetate and passed through a short plug of silica or alumina, eluting with ethyl acetate. The liquor is then condensed and repeated until precipitation stops, and the remaining crude product is purified by flash chromatography. The combined products are dried under vacuum to afford the desire b-hydroxy ester in 75-98% yield.

### Process Examples

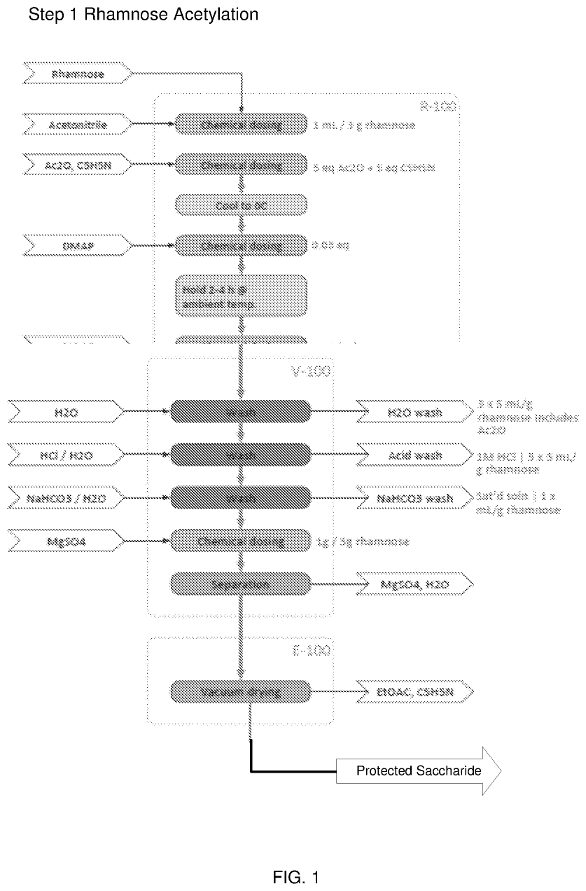

Turning now to FIGS. 1-5, five example process steps are illustrated. Each step 1-5 is described as having sub steps, which may also be referred to as “steps”. In FIG. 1, an embodiment of a process step 1 is illustrated. In irhamnose is dissolved in Acetonitrile in a reaction vessel (R-100). In one embodiment, 1 mL of Acetonitrile is added per 3 grams of rhamnose. Then acetodenhydride (5 eq Ac2O plus 5 eq C5H5N) is added. In one embodiment, the Ac2O and C5H5N is cooled as its added. The mixture may be cooled to 0° C. A catalyst may then be added. In one embodiment, the catalyst is DMAP at 0.03 eq. This mixture is stirred at ambient temperature using the stirring techniques described herein throughout. The mixture may be stirred for up to 2 hours of more until it is determined that the reaction is substantially complete. In other embodiments, the mixture may be stirred for up to 4 hours or more. In one embodiment, mixture is stirred at least about 2 hours. In one embodiment, the mixture is stirred less than about 4 hours. The completeness of the reaction may be checked using thin layer chromatography (TLC). Once the reaction is done, EtOAC may be added. In one embodiment, 1 mL of EtOAC per gram of rhamnose may be added.

The resulting mixture may be transferred to mixer, blender, or other post-reaction vessel device (V-100) where water is added to the mixture. The mixture may be stirred to allow for separation and then the water may be removed. This water wash may be done multiple times. In one embodiment, 5 mL of water per gram of rhamnose may be added with the water then being removed. Next HCl and water may be added for an acid wash with the acid wash then being removed. This step may be repeated multiple times. In one embodiment, 5 mL of 1 M HCL per gram of rhamnose is added and stirred to allow for separation. Then the acid wash is removed. The acid wash step is repeated three times in one embodiment. The resulting mixture is then given a sodium bicarbonate wash where NaHCO₃ and water is added to the mixture. The mixture is stirred an allowed to separate and the NaHCO₃wash is removed. In one embodiment, one mL of saturated solution of NaHCO₃ per gram of rhamnose is added. In this embodiment, the NaHCO₃wash is not repeated. The washing may leave a mixture of protected sugar and the ethyl acetate. One gram of MgSO₄ per 5 grams of rhamnose may be added to the washed mixture and the MgSO₄ and water may be separated out. In one embodiment, the MgSO₄ is filtered out of the mixture.

The resulting mixture may be transferred to a dryer (E-100). The dryer may be a blender, an evaporator, or other dryers known in the art. In one embodiment, the mixture is vacuum dried. In other embodiments, the drying is done in the mixer, blender, or post reaction vessel device (V-100). Once the mixture is partially or substantially dried, the solvent may be removed under reduced pressure. In one embodiment, EtOAC and C5H5N is removed at this step leaving an amount of protected saccharide. The protected saccharide may be used later as will be described below.

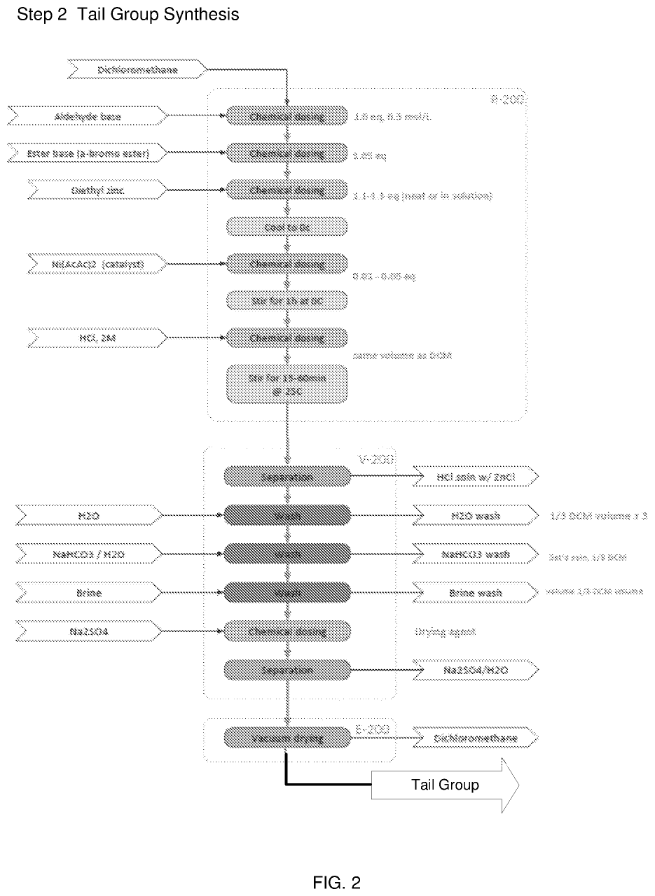

Turning now to FIG. 2, an embodiment of a process step 2 is illustrated. In this process step, dichloromethane (DCM) may be added to a rounded flask (R-200). Next, reactants may be added. In one embodiment, 0.3 mol per liter of Aldehyde base (1.0 eq) and Ester base (a-bromo ester) (1.05 eq) are added. In one embodiment, Diethyl zinc may be added at 1.1-1.3 eq (neat or in solution). The mixture may then be cooled to about 0° C. After cooling, a catalyst may be added. In one embodiment the catalyst is Ni(AcAc)², which may be added at 0.01-0.05 eq, for example. This mixture may then be stirred for about 1 hour at about 0° C. The completeness of the reaction may then be checked using thin layer chromatography. Material to stop any further reaction may then be added. In one embodiment, 2M HCl is added. In one embodiment, the volume of added material is the same as the volume of dichloromethane used. The mixture may be stirred for about 15 minutes at up to 25° C. In one embodiment, mixture is stirred at least about 15 minutes of stirring. In one embodiment, the mixture is stirred less than about 120 minutes. In one embodiment, the added material may be stirred for up to 60 minutes and beyond at up to 25° C.

The mixture may be transferred to mixer, blender, or post reaction vessel device (V-200) where the HCL solution with ZnCl is separated out. The remaining mixture may then be washed with a water wash and/or a NaHCO₃ wash. The water wash may be repeated several times. In one embodiment, the volume of water is one third the volume of DCM used for a water wash. The water wash is removed and the water wash step is repeated three times. The NaHCO₃ wash may include adding a volume of saturated NaHCO₃ in an amount of one third the amount of DCM used. In one embodiment, a brine wash may be used as well. The brine wash may facilitate drying the mixture. In one embodiment the volume of brine used is one third the amount of DCM used. After the washing, a drying agent such as Na₂SO₄ or other drying agent may be added to the mixture. The Na₂SO₄/H₂O is then separated out of the mixture.

In one embodiment of Step 2, the mixture may then be transferred to a dryer, which may be a rotary evaporator. In one embodiment, the mixture is vacuum dried and Dichloromethane is removed to leave the desired tail group. The tail group may be used later in the process as described below.

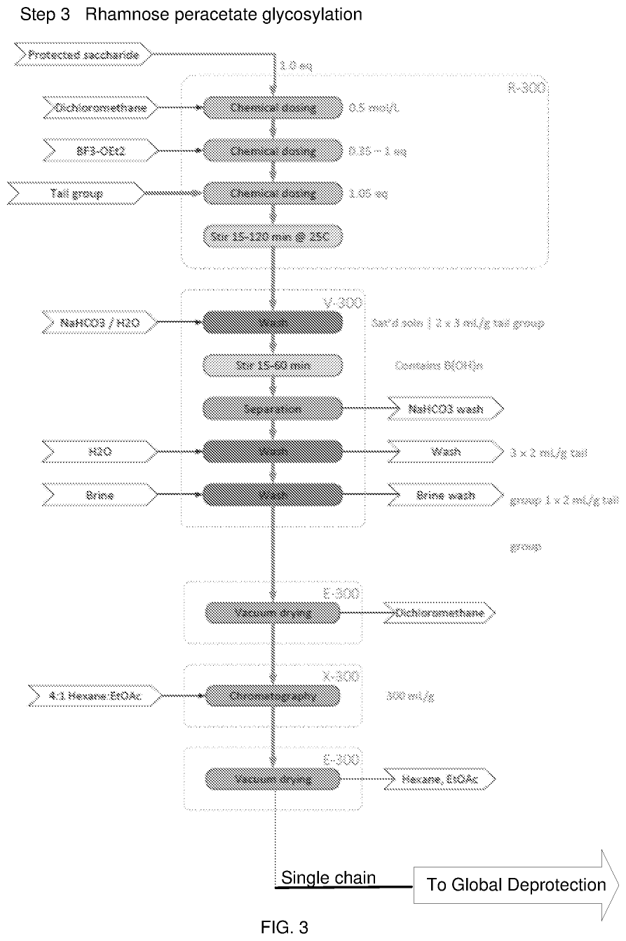

Turning now to FIG. 3, an embodiment of a process step 3 is illustrated. The protected saccharide of Step 1 (1.0 eq for example) is added to a reactor (R-300). The protected saccharide may be dissolved. DCM may be added to facilitate dissolving in amount of 0.5 mol/L. In one embodiment, a glycosylation promoter of BF₃₋OEt₂ (about 0.35 eq to about 1 eq) is added to the mixture. The tail group from Step 2 may then be added (about 1.05 eq) to the mixture. The mixture is allowed to react. The reaction is facilitated by at least about 15 minutes of stirring. In one embodiment, the mixture is stirred less than about 120 minutes. The stirring may be done at up to 25° C. The completion of the reaction may be checked by ways known in the art, including TLC.

The mixture may then be transferred to a mixer, blender, or post reaction vessel device (V-300) where the mixture is neutralized, separated, and washed. The neutralizer material may be NaHCO₃ in a wash. In one embodiment, a saturated solution of NaHCO₃ in an amount of 3 mL per gram of tail group is used. The neutralizer washing may be repeated multiple times. In one embodiment, the step is repeated twice. The mixture may be stirred for at least about 15 minutes. In one embodiment, the mixture is stirred for less than about 60 minutes. After stirring, the NaHCO₃wash may be separated out of the mixture. The remaining mixture may then be washed with water and/or brine. In one embodiment, 2 mL of water per gram of tail group is used for the water wash. The water wash is then removed from the mixture. This water wash step may be done multiple times. In one embodiment, 2 mL of brine per gram of tail group may be used for the brine wash. The brine wash may then be removed from the mixture. In one embodiment, the brine wash is only done once.

The mixture may then be transferred to a drying (E-300) where it is dried according to ways know in the art. In one embodiment, multiple drying steps are used. In one drying step, DCM is removed. A chromatography step may be done where 4 parts Hexane to 1 part EtOAc is added to the mixture. A reaction status may be checked at this time. The Hexane and EtOAc may then be removed in a subsequent drying step. The remaining mixture may be a desired single chain product that includes a glycolipid.

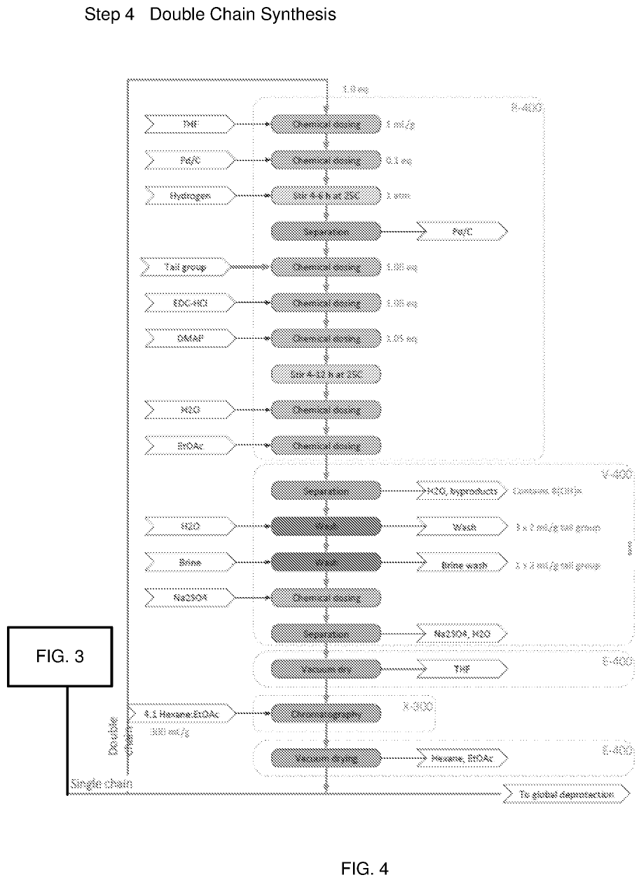

Turning now to FIG. 4, an embodiment of a process step 4 is illustrated. It may be desired to have a double chain product. If so, Step 4 may be used on the resulting single chain mixture of Step 4. In Step, a blocking group may be removed from one half of the single chain glycolipid molecule existing after Step 3. In one embodiment, this is accomplished by adding THL as a solvent in an amount of 1 mL per gram. Then Pd/C may be added to the mixture which is then stirred. In one embodiment, the mixture is stirred in an atmosphere of hydrogen gas. The mixture may be stirred at least 4 hours in this atmosphere. In one embodiment, the mixture is stirred less than 6 hours in the hydrogen atmosphere. Once the resulting reaction is completed, as may be checked by TLC, the Pd/C is removed from the mixture by simple filtration. A tail group is then added to the resulting mixture.

In one embodiment the tail group of Step 4 does not have a sugar added to it. In another embodiment, the tail group is not the tail group that is used in Step 3. A tail group (1.05 eq) may be added. A coupling agent may be added to the mixture. In one embodiment, the coupling agent is EDC-HCl (1.05 eq). A catalyst may be added to the mixture. In one embodiment, the catalyst is DMAP (1.05 eq). The mixture may then be stirred to facilitate the reaction of the constituents. In one embodiment the mixture is stirred by stirring techniques known in the art for at least about 4 hours. In another embodiment, the mixture is stirred for less than about 12 hours. The mixture may be stirred at up to 25° C. When the reaction is complete, which may be checked by ways known in the art, including THL, water and EtOAc may be added to the mixture. In one embodiment, the tail group of Step 4 may attach to the resulting molecule conjugate of Step 3 where the tail group of Step 3 attached to the resulting molecule conjugate of Step 1 and/or 2. Stated another way, the result of Step 3 is a sugar with one tail group. The result of Step 4 is another tail group added to the sugar with the first tail group.

The mixture may then be transfer to a mixer, blender, or post reaction vessel device (V-400) where water and by products may be removed. The remain mixture may be washed by ways described herein throughout or other ways known in the art. In one embodiment, 2 mL of water per gram of Step 4 tail group is used. In other embodiments, 2 mL of brine per gram of Step 4 tail group is used. The washing sub steps may be repeated more than one time. A drying agent may then be added. In one embodiment, Na₂SO₄ is added to the mixture. Water and Na₂SO₄ may then be removed. The resulting mixture may then be purified using sub steps described in connection with Step 3 including removing THF by drying, performing 4 parts to 1 of Hexane to EtOAc, and removal of the Hexane and EtOAc by further drying. The resulting mixture yields a double chain glycol lipid.

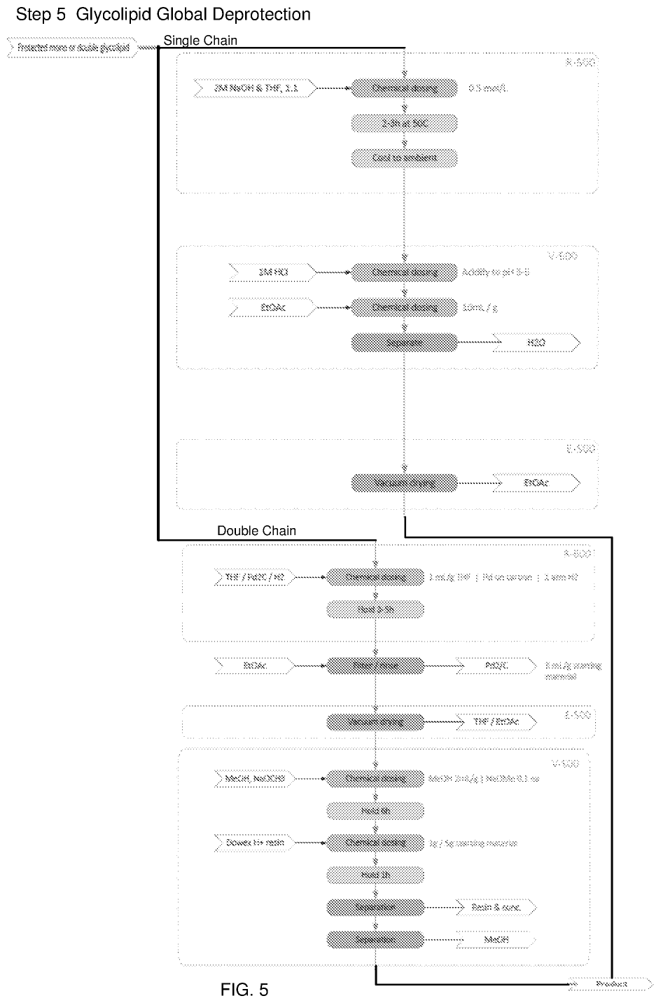

Turning now to FIG. 5, an embodiment of a process step 5 is illustrated. In this step, the sugar protection of single chain and/or double chain glycolipids are removed. For a single chain glycolipid, the single chain glycolipid is dissolved in a one to one mixture of 2M NaOH and THF in a reaction vessel (R-500). The amount of this mixture may be 0.5 mol/L. The mixture may then be heated. In one embodiment, the mixture is heated for more than 2 hours. In another embodiment, the mixture is heated less than 3 hours. The mixture may be heated up to about 50° C. After heating, the mixture may then be cooled to ambient and transferred to mixing or suitable post reaction vessel (V-500). The mixture may then be neutralized using 1M HCL. In one embodiment 1M HCL is added until the mixture has an acidity of more than about pH 3. In another embodiment, HCL solution is added until the mixture has an acidity of less than about pH 5. EtOAc may be added to help separate water from the mixture. EtOAc may then be removed by drying or other appropriate steps to get the desired single chain glycolipid product.

For double chain glycolipids, glycolipid global deprotection is achieved by adding THF, Pd₂C, and H₂ in a suitable reaction vessel (V-500). In one embodiment, 1 mL of Palladium on Carbon per gram of THC is added in 1 atmosphere of Hydrogen. The resulting reaction is allowed to go for at least about 3 hours. In one embodiment, the reaction to allowed to proceed for less than about 5 hours. Once the reaction is complete, as determined by methods described herein throughout, the Palladium on Carbon (Pd₂/C) is separated out by adding EtOAc to the mixture. In one embodiment, 3 mL of EtOAc is added per gram of starting material. A suitable drying apparatus (E-500) may be used to remove the solvent (THF/EtOAc) from the mixture. The resulting mixture or product may be dissolved in a suitable vessel (V-500). The solvent may be MeOH with NaOCH₃ as an active agent to facilitate leaving esters on the sugar of the mixture molecule. In one embodiment, 2 mL of MeOH per gram of NaOCH₃ may be added to the mixture to form NaOMe. The dissolving step may take up to about 6 hours in one embodiment.

Dowex H, a solvent form of acid, and resin may be added to neutralize the MeOH, and/or quench any further reaction. In will be appreciated that other forms of acid neutralizer may be used including without limitation acid/water solutions. In one embodiment, 1 gram of Dowex H plus resin per 5 grams of starting material may be used. The mixture may be allowed to stand for up to about 1 hour in certain embodiments. The resin and undesired reaction byproducts and MeOh may be separated out from the mixture in one or more sub steps to achieve the desired double chain glycolipid product.

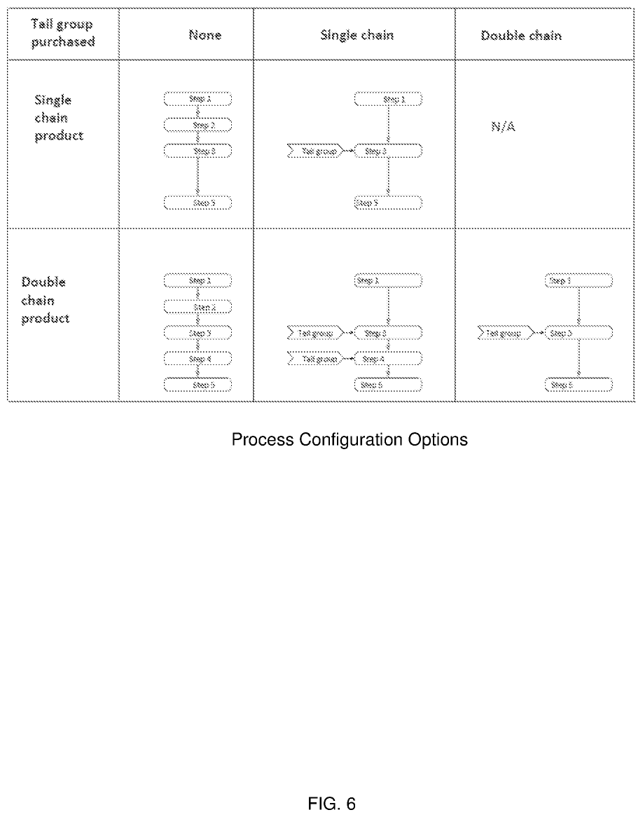

Turning now to FIG. 6, a chart showing process step configurations is shown. The chart shows step combinations of tail groups purchased (no purchase, single chain tail group purchased, and double chain tail group purchased) to create single chain products and double chain products. Where no tail group was purchased, a single chain product may be accomplished using Step 1described above, followed by Step 2 described above, followed by Step 3 described above, followed by Step 5 described above. Where a single chain tail group is purchased or used to make a single chain product, Step 1 described above may be followed by Step 3, where the single chain tail group is added, followed by Step 5. Where a double chained product is desired without using a purchased tail group, Steps 1-5 described above may be used. Where a double chained product is desired using a purchased single chain tail group, Step 1 may be used, followed by Step 3, where the purchased tail group is added, followed by Step 4, where the purchased tail group is added, followed by Step 5. Where a double chained product is desired using a double chain purchased tail group, Step 1 described above may be followed by Step 3, where the single chain tail group is added, followed by Step 5.

### Specific Example Procedure. Beta Hydroxy Ester (Tail Group Synthesis)

To a 20 L round-bottom flask equipped with a mechanical stirrer, was added 216 mL (1.15 mol, 1 equiv.) decanal, 238 mL (1.5 mol, 1.3 equiv.), benzyl bromoacetate, and 3 L dichloromethane (0.4M). The reaction mixture was sparged with N₂ with stirring for 15 minutes and maintained under a nitrogen atmosphere. To this mixture was added 1.5 L diethylzinc (1M in hexane, 1.5 mol, 1.3 equiv) under a nitrogen atmosphere by cannula transfer. The resulting mixture was then cooled in an ice-bath to an internal temperature below 5° C. To the cooled solution was added 19 g Ni(AcAc)₂ (0.08 mol, 0.05 equiv.) in a single portion under a stream of nitrogen. The resulting solution heated up to ˜30° C. within 2 min, followed by cooling. Reaction progress was monitored by TLC. Upon completion of the reaction, 2 L of 2M HCl was added slowly, and the biphasic mixture was stirred for 15 min. The mixture was then added to a separatory funnel, where the aqueous phase was discarded, and the organic layer was washed successively with H₂O, sat'd NaHCO₃, and brine followed by drying over Na₂SO₄. The solvent was removed to afford a viscous yellow oil that was then dissolved in 500 mL hexane and placed in a −40° C. freezer. The precipitated white crystalline solid was washed with minimal −40° C. hexanes. The solid is then dried under vacuum. Yielding 168 g (84%, 2 precipitations) of b-hydroxy ester as a low-melting waxy solid.

The reaction for the beta hydroxy ester tail group synthesis described above is as follows:

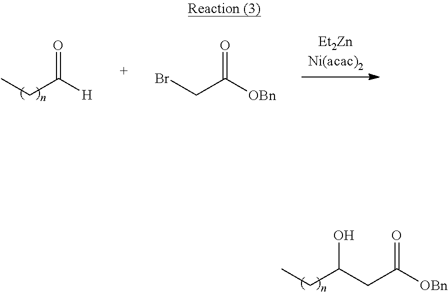

The yield ranges for the formation of b-hydroxy benzyl esters from their respective aldehyde starting materials are shown in Table 1 below.

TABLE 1

| aldehyde | form | yield |
|---|---|---|
| butanal | oil | 72-89% |
| hexanal | oil | 66-80% |
| octanal | oil | 64-84% |
| decanal | solid | 72-89% |
| dodecanal | solid | 75-91% |
| hexadecanal | solid | 54-70% |

### Specific Example Procedure. Glycosylation

To 1 L round-bottom flask was added 60.0 g C10-b-hydroxy ester (215.5 mmol, 1.1 equiv), 65 g peracylated rhamnose (196 mmol, 1.0 equiv) and 250 mL dry dichloromethane (˜1M). The resulting solution was cooled to 0° C., and 27 mL BF₃₋OEt₂ (215.52 mmol, 1.1 equiv). The solution was removed from the ice bath, and reaction progress was monitored by TLC. Upon completion of the reaction 500 mL sat'd NaHCO₃ was added and stirred until neutralization (˜20 min). The mixture was then added to a separatory funnel, where the aqueous phase was discarded, and the organic layer was washed successively with H₂O and brine followed by drying over Na₂SO₄. The solvent was removed to afford a viscous yellow oil that was purified by column chromatography (silica, 33% EtOAc/Hexane) yielding 82 g glycosylated product (76%) as a viscous light-yellow oil.

The reaction for the glycosylation procedure described above is as follows:

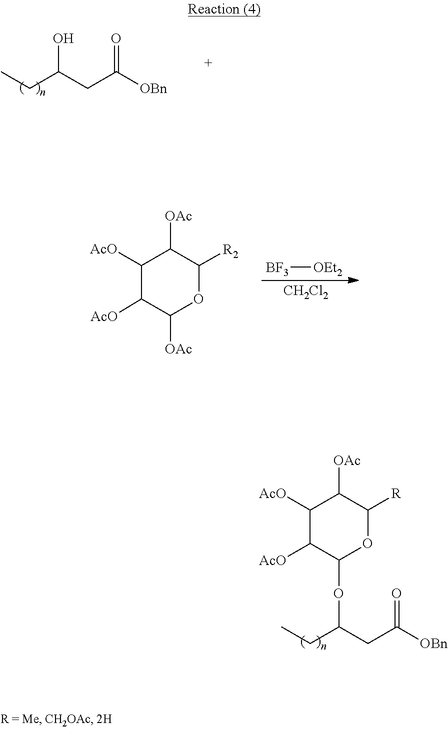

The yield ranges for the glycosylation of b-hydroxy benzyl esters with various peracylated sugars is shown in Table 2 below:

TABLE 2

| β-hydroxy | | |
|---|---|---|
| ester | Sugar | yield |
| n = 2 | rhamnose | 71% |
| n = 6 | rhamnose | 68% |
| n = 10 | rhamnose | 82% |
| n = 10 | xylose | 67% |
| n = 10 | galactose | 54% |

In certain embodiments, the process is used to make products, including surfactants, beta hydroxy esters as tail chains, for example. The products may be intermediates or stand-alone products.

The processes of the present invention overcome some problems with prior art processes in that embodiments are solution based. Accordingly, they are less time consuming and less expensive than non-solution phase processes. Additionally, the glycosylation processes of the present invention is performed at reduced temperature ranges. Often around room temperature. This reduces unwanted reactivity and increases glycolipid yield. The present invention processes also use a smaller amount of glycosylation promotor than other processes so there is less reaction, which also increases yield. Because the processes of the present invention use systems, process conditions, and materials that allow for a more control, less reactive process, glycolipid yield is higher. Additionally, systems, process conditions, and materials of the present invention allow for slower processing, which also allows for more process control, and thus higher glycolipid yields.

While certain illustrative embodiments have been described in connection with the figures, those of ordinary skill in the art will recognize and appreciate that embodiments encompassed by the disclosure are not limited to those embodiments explicitly shown and described herein. Rather, many additions, deletions, and modifications to the embodiments described herein may be made without departing from the scope of embodiments encompassed by the disclosure, such as those hereinafter claimed, including legal equivalents. In addition, features from one disclosed embodiment may be combined with features of another disclosed embodiment while still being encompassed within the scope of embodiments encompassed by the disclosure as contemplated by the inventors.

The scope of the present invention is defined by the appended claims.

APPENDIX A

| Monosaccharides | Disaccharides | Trisaccharides |
|---|---|---|
| allose | cellobiose | cellotriose |
| altrose | chitobiose | isomaltotriose |
| arabinose | dirhamnose | isopanose |
| fructose | gentiobiose | laminaritriose |
| fucose | isomaltose | manninotriose |
| galactose | isomaltulose | maltotriose |
| glucose | lactose | melezitose |
| gulose | lactulose | nigerotriose |
| idose | laminaribose | panose |
| lxyose | leucrose | raffinose |
| psicose | maltose | xylotriose |
| rhamnose | maltulose | |
| ribose | melibiose | |
| 2-deoxy-ribose | nigerose | |
| ribulose | sophorose | |
| sorbose | sucrose | |
| tagatose | terhalose | |
| talose | turanose | |
| xylose | xylobiose | |
| xylulose | | |

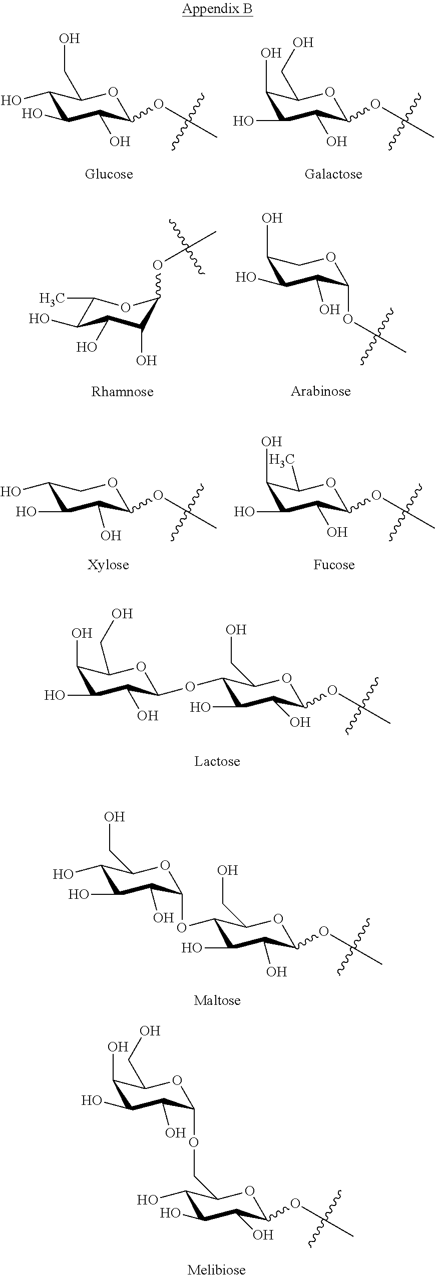

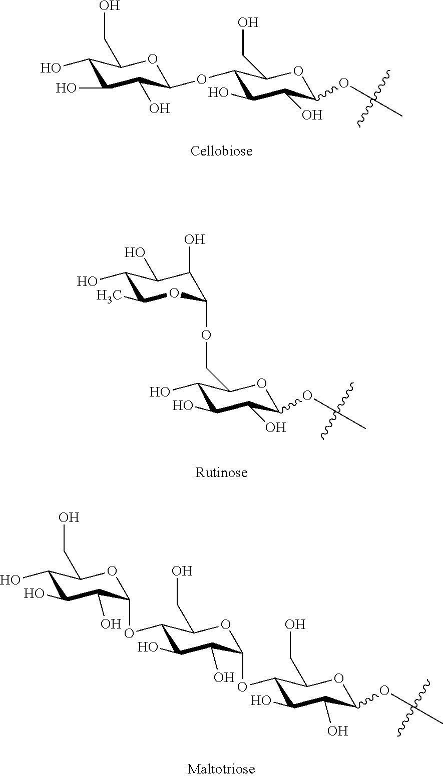

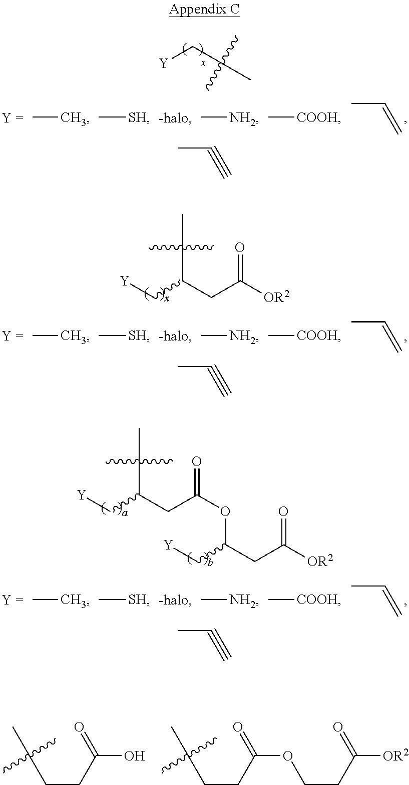

What is claimed is:

1. A method of forming a glycolipid, comprising:
Glycosylating a sugar with a beta-hydroxyester using BF3-L as a glycosylation promoter.

2. The method of claim 1, wherein L comprises one or more of Diethyl ether (Et₂O), Tetrahydrofuran (THF), Dimethyle Sulfide (Me₂S), or combinations thereof.

3. The method of claim 1, wherein the beta-hydroxyester comprises a carbon length ranging from C2 to C24.

4. The method of claim 3, wherein the beta-hydroxyester comprises a carbon length ranging from C8 to C18.

5. The method of claim 4, wherein the beta-hydroxyester comprises a carbon length ranging from C10 to C16.

6. The method of claim 1, wherein the beta-hydroxyester comprises a natural beta-hydroxyester.

7. The method of claim 1, wherein the beta-hydroxyester comprises a synthetic beta-hydroxyester.

8. The method of claim 1, wherein the sugar comprises a protected sugar.

9. The method of claim 1, wherein the sugar comprises one or more of peracetylated-rhamnose, peracetylated-xylose, peracetylated-glucose, peracetylated-galactose or combinations thereof.

10. The method of claim 1, wherein the beta-hydroxyester comprises the formula: 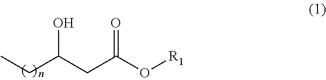
and wherein R₁ comprises one or more of a methyl functional group (Me), an ethyl functional group (Et), a benzyl functional group (Bn), a methoxymethyl functional group (MOM), a methoxyethoxymethyl functional group (MEM), or combinations thereof.

11. The method of claim 1, wherein the beta-hydroxyester comprises the formula: 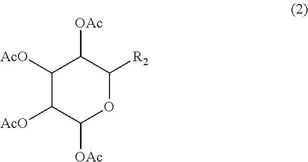
and wherein R₂ comprises one or more of heavy hydrogen (2H), a methyl functional group (Me), a CH₂OAc group, or combinations thereof.

12. The method of claim 1, wherein glycosylating a sugar with a beta-hydroxyester using BF3-L as a glycosylation promoter comprises a stoichiometric reaction wherein the sugar has a molar equivalent ranging from about 1.1 to about 1.2.

13. The method of claim 1, wherein glycosylating a sugar with a beta-hydroxyester using BF3-L as a glycosylation promoter comprises a stoichiometric reaction wherein the beta-hydroxyester has a molar equivalent of about 1.

14. The method of claim 1, wherein glycosylating a sugar with a beta-hydroxyester using BF3-L as a glycosylation promoter comprises a stoichiometric reaction wherein the BFR-L has a molar equivalent ranging from about 0.33 to about 1.

15. A method of forming a glycolipid, comprising:
mixing a b-hydroxyester with a peracetylated sugar in a reaction vessel to form a mixture;
purging the reaction vessel comprising the mixture to create an inert atmosphere within the reaction vessel;
adding a solvent to the mixture to create a solution wherein the mixture is substantially dissolved in the solvent;
adding BF₃₋ L to the solution, wherein the step of adding BF₃₋ L to the solution is performed such that solution temperature is maintained within a desired temperature range.

16. The method of claim 15, wherein the b-hydroxyester comprises the molecule: 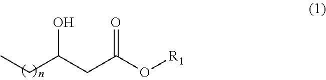
and wherein the b-hydroxyester comprises an equivalent of about 1 in the mixing reaction.

17. The method of claim 15, wherein the peracetylated sugar comprises the formula: 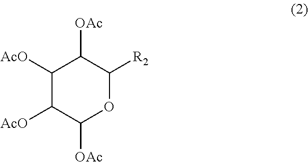
and wherein the peracetylated sugar comprises an equivalent of about 1.1 to about 1.2 inclusive.

18. The method of claim 15, wherein the solvent comprises dry dichloromethane.

19. The method of claim 15, wherein the desired temperature range is between about 13° C. and about 33° C.

20. The method of claim 15, wherein the desired temperature range is about room temperature.

21. The method of claim 15, wherein adding BF₃₋ L to the solution comprises stirring the BF₃₋L and the solution until at least 70% of the b-hydroxyester is consumed.

22. The method of claim 15, wherein adding BF₃₋ L to the solution comprises stirring the BF₃₋L and the solution in an inert atmosphere for at least about 5 hours.

23. The method of claim 15, further comprising diluting the mixture of BF₃₋ L and solution.

24. The method of claim 23, wherein diluting the mixture of BF₃₋ L and solution comprises doubling the volume of dichloromethane used in a prior step.

25. The method of claim 24, further comprising adding saturated sodium bicarbonate to the diluted mixture of BF₃₋ L and solution to quench the reaction within the reaction vessel.

26. The method of claim 25, further comprising removing an aqueous layer from the remaining solution.

27. The method of claim 26, further comprising washing the remaining solution with deionized water.

28. The method of claim 27, further comprising adding a brine solution to the washed remaining solution.

29. The method of claim 28, further comprising drying the remaining solution.

30. The method of claim 29, further comprising purifying the remaining solution.

31. The method of claim 30, wherein the purified remain solution comprises a glycolipid yield of between about 65% and about 95%.
